(19) United States
(12) Patent Application Publication
(10) Pub. No.: US 2007/0224261 A1
(43) Pub. Date: Sep. 27, 2007

(54) EUTECTIC LIQUID DRUG FORMULATION

(76) Inventor: Peter Draper, Windsor (CA)

Correspondence Address: GIFFORD, KRASS, SPRINKLE,ANDERSON & CITKOWSKI, P.C, PO BOX 7021, TROY, MI, 48007-7021, US

(21) Appl. No.: 11/684,666

(22) Filed: Mar. 12, 2007

Related U.S. Application Data
(60) Provisional application No. 60/784,673, filed on Mar. 22, 2006.

(51) Int. Cl. A61K 9/66

(52) U.S. Cl. 424/452

(57) ABSTRACT

A drug formulation dosage is provided that includes an oral medication capsule inclusive of the eutectic liquid at room temperature. The eutectic liquid includes a pharmaceutically active substance that exists as a solid in pure form at room temperature along with a biologically tolerated compound that forms a eutectic liquid with the active substance so as to render the substance and the compound as the eutectic liquid. The resulting drug formulation dosage has a high degree of storage stability. Natural volatile oils represent a class of biologically tolerated compounds well suited for the formation of a eutectic liquid sealed within an oral medication capsule. Three or more component eutectics are also appreciated to be operative herein to form stable eutectic liquids with pharmaceutically active substances. A process for delivering a pharmaceutically active substance existing at room temperature in pure form as a solid as an oral dosage includes administering to a subject orally a pharmaceutically effective amount of the substance as a eutectic liquid formed with the biologically tolerated natural volatile oil. The eutectic liquid is contained within a capsule disintegrating subsequent to administration to the subject and thereby releasing the pharmaceutically active substance. A transdermal medicament delivery system is also provided that includes multiple small capsules of a drug formulation dosage as detailed above. The integrity of the capsule is compromised after exposure to subject skin through temperature, perspiration dissolution or concussion. A skin compatible adhesive matrix retains the multiple drug formulation dosage capsules in contact with subject skin so as to release the active substance into contact with subject skin.

### CROSS-REFERENCE TO RELATED APPLICATIONS

This application claims priority of U.S. Provisional Patent Application Ser. No. 60/784,673 filed Mar. 22, 2006, which is incorporated herein by reference.

### FIELD OF THE INVENTION

The present invention in general relates to a pharmaceutically active substance or substances solid in pure form at room temperature nixed with a biologically tolerated compound or compounds to form a eutectic liquid at room temperature and in particular to such a drug formulation delivered orally or topically within a capsule.

### BACKGROUND OF THE INVENTION

A suitable drug formulation has many criteria including shelf stability, acceptable form and size for administration, manufacturing convenience and effective therapeutic effect. Drug substances are formulated into pharmaceutically acceptable dosage forms such as tablets, capsules and oral liquids for oral administration. Other forms of administration are widely published including creams, pastes, sprays and solutions. Unfortunately, many drug substances have adverse chemical or physical properties that lead to manufacturing, stability or bioavailability problems. One established method of delivering such a drug substance is in the form of a soft gel capsule. Drug formulation strategies for the fill materials of soft gel capsules are widely published and include hydrophilic or lipophilic solutions, suspensions or emulsions containing the drug substance. In the simplest form, an active compound is delivered as a liquid formulation as a syrup or solution, or as a liquid encompassed within a gelatin capsule. Typical solvents used to solubilize an active substance include dimethyl acetamide, dimethyl sulfoxide, diethylene glycol ethers, polyglycols, castor oil derivatives and combinations thereof, alone or in combination with surfactants. While solubilized forms of active substances retain therapeutic efficacy, such soluble formulations have limited storage stability owing to temperature-dependent changes in solubility that can result in the active substance precipitating from the solution. Additionally, such solutions tend to contain a comparatively small percentage of active substance, meaning that a large volume capsule or liquid formulation is required. Since a number of solvents and/or active substances are not particularly palatable, the resulting drug formulation as a syrup, drop or solution is highly unpleasant to ingest thereby compromising subject compliance.

Other formulation techniques available include micronization of a powder or the formation of emulsions and/or microemulsions. Unfortunately, these techniques likewise create problems associated with production and stability. Additionally, in instances where an additive such as an emulsifier or a solvent is added, in addition to incurring stability problems, the additive also increases the volume that must be contained with a capsule or tablet thereby making ingestion all the more difficult for a subject.

Still another approach to drug delivery includes the formation of a eutectic involving the active substance. In the event that a eutectic mixture is formed that has a lower melting point than the active substance, it is likely that many of the aforementioned problems associated with drug delivery could be overcome, particularly if such eutectic mixtures are contained in soft gel capsules. There are teachings in the literature to show stability problems may be encountered with conventional solid dosage forms as detailed in U.S. Pat. No. 5,512,300 concerning the formation of eutectics in tablet formulations. Soft gel capsules are compatible with eutectic mixtures of active substances and avoid such stability problems.

Successful topical formulations have been produced that include an active substance as part of a liquid eutectic mixture. By way of example, topical eutectic mixtures include ibuprofen-methyl nicotinate (U.S. Pat. No. 6,841,161 B1), 4:96 ibuprofen:menthol (weight percent/weight percent) (WO 91/04733), 40:60 ibuprofen:thymol (weight percent/weight percent) (Stott et al., J. Controlled Rel. 50:297-308 (1998)), and 20:80-58:42 lidocaine:prilocaine (weight percent/weight percent) (U.S. Pat. No. 4,562,060). Other examples of eutectics are: 25:75 ibuprofen:racemic menthol (weight percent/weight percent) 13° C. and 30:70 ibuprofen:L-menthol (weight percent/weight percent) 19° C. (Stott et al.). Unfortunately, such eutectic mixtures have not been contemplated previously for oral delivery drug formulations.

There exists a need for a eutectic liquid containing a pharmaceutically active substance otherwise solid in pure form at room temperature forming an oil with a biologically tolerated compound, the resulting eutectic mixture being amenable to encapsulation.

### SUMMARY OF THE INVENTION

A drug formulation dosage is provided that includes an oral medication capsule inclusive of the eutectic liquid at room temperature. The eutectic liquid includes a pharmaceutically active substance that exists as a solid in pure form at room temperature along with a biologically tolerated compound that forms a eutectic liquid with the active substance so as to render the substance and the compound as the eutectic liquid. The resulting drug formulation dosage has a high degree of storage stability. Natural volatile oils represent a class of biologically tolerated compounds well suited for the formation of a eutectic liquid sealed within an oral medication capsule. Three or more component eutectics are also appreciated to be operative herein to form stable eutectic liquids with pharmaceutically active substances.

A process for delivering a pharmaceutically active substance existing at room temperature in pure form as a solid as an oral dosage includes administering to a subject orally a pharmaceutically effective amount of the substance as a eutectic liquid formed with the biologically tolerated natural volatile oil. The eutectic liquid is contained within a capsule disintegrating subsequent to administration to the subject and thereby releasing the pharmaceutically active substance.

A transdermal medicament delivery system is also provided that includes multiple small capsules of a drug formulation dosage as detailed above. The integrity of the capsule is compromised after exposure to subject skin through temperature, perspiration dissolution or concussion. A skin compatible adhesive matrix retains the multiple drug formulation dosage capsules in contact with subject skin so as to release the active substance into contact with subject skin.

### DETAILED DESCRIPTION OF THE PREFERRED EMBODIMENTS

The present invention has utility as an encapsulated drug formulation dosage containing an active substance in the form of a eutectic liquid at or near to room temperature. As used herein, “at or near to room temperature” is defined to mean a temperature range of 15 to 40° Celsius.

A “eutectic” as used herein is defined to include a system containing at least one active substance and at least one other biologically tolerated compound that displays on a plot of melting temperature versus relative composition at least one minimum point associated with a homogeneous liquid phase, such a point synonymously denoted as a eutectic point or eutectic temperature.

The inventive eutectic liquid pharmaceutically active substance that is solid in pure form at room temperature includes any substance used in the prevention or therapy of a condition affecting the health of a mammal, including a human. The pharmaceutically acceptable substances solid in pure form at room temperature operative herein illustratively include antimicrobial agents such as mupirocin, triclosan, chlorocresol, chlorbutanol, iodine, clindamycin, ketoconazole, and econazole; anti-inflammatory analgesic compounds such as acetylsalicylates, salicylates, choline salicylates, opioid analgesics such as fentanyl; rubefacients such as capsaicin; arylpropionic acid derivatives such as ibuprofen, ketoprofen, fenoprofen and flurbiprofen; aryl acetic acid derivatives such as etodolac; methyl nicotinate; anti-motion sickness agents such as scopolamine; antihistamines such as triprolidine and promethazine; antihypertensives such as propranolol; antispasmodic agents such as oxybutynin; anthelminthics such as levamisole and tetramisole; as well as vitamins, minerals and other nutrients.

Eutectic-forming topical pharmaceutically active substances are well known in the art as embodied in U.S. Pat. No. 6,841,161. Upon selecting a desired pharmaceutically active substance that is compatible with a conventional capsule employed in oral delivery of pharmaceuticals, a biologically tolerated compound is chosen that forms a eutectic liquid at or near to room temperature upon mixing with the pharmaceutically active substance.

The formation of a satisfactory eutectic requires a selection of at least one ingestible compound interactive with a pharmaceutically active substance and compatible with an oral delivery capsule. A eutectic forming compound preferably is a terpene. More preferably, the eutectic-inducing compound is a volatile oil. Illustrative examples of purified components found within a volatile oil that appear to form eutectics with a variety of pharmaceutically active substances illustratively include: menthol, thymol, eugenol and carvacrol. A key feature of eutectics formed according to the present invention is that stable phases present within the dosage form at a rate that keeps the melt composition constant. As such, eutectics are most likely to form as complexes of the form AB, AB₂, or A₂B where A is a pharmaceutically active substance and B is a biologically tolerated compound.

Thermodynamically, the formation of eutectic complexes and eutectic forming mixtures have in general positive increases in kinetic energy relative to increases in potential energy. As such, excess enthalpy, excess Gibbs energy and excess entropy.

Thermodynamic treatment of mixtures capable of forming stable complexes assuming ideal behavior is provided by the equation:

$$\frac{{Δ}_{f} h}{R} ( \frac{1}{T} - \frac{1}{{T}_{c}} ) = - = {ln ( {ξ}_{A}^{I} )}^{m} {( {ξ}_{B}^{1} )}^{n} + {ln ( {ξ}_{A}^{1} )}_{c}^{m} {( {ξ}_{C}^{1} )}^{n} \qquad ( 1 )$$

where the compound formed is AₘBₙ·ξ_{A}¹ and ξ_{B}¹ are the mole fraction of the species A and B, respectively. These would be equal to the stoichiometric mole fractions x_{A} and x_{B} if the compound is completely dissociated in the molten state. Subscript C represents the corresponding quantity at the congruent melting point T_{C}. Superscript 1 denotes the liquid phase. An approximate idea of heat of fusion of the complex can be obtained from Equation 1 by assuming complete dissociation of the complex and plotting (1/T−1/T_{C}) against 1n(x_{A}¹)ⁿ(x_{B}¹)ⁿ. (R. P. Rastogi, Pure & Appl. Chem. 66(3):441-448 (1994)).

Experimental studies to indicate the formation of complexes are readily performed by polarized Rayleigh light scattering or static Kerr effect. A common feature of eutectic formation according to the present invention is the presence of hydrogen bonding or donor acceptor interaction associated with an AₙBₘ complex associated with a eutectic composition where n is an integer 1 or 2, and m is an integer 1 or 2. As such, a configuration should exist for a stable complex associated with a eutectic that involves a strong interaction between an electron donor group within one of the two components A or B such as a carbonyl oxygen, amidyl nitrogen, or etheral oxygen with alkyl proton, hydroxyl proton, amine proton or a sigma acceptor group such as chlorine.

Without intending to be bound by a particular theory, it is believed that an inventive drug formulation dosage eutectic liquid forms stable A-B type complexes through parallel stacking of molecules. More particularly, the parallel stacking of molecules in maintaining an interplanar distance of between 3 and 3.4 angstroms is believed to provide for optimal hydrogen bonding or dipole-dipole interaction for eutectic formation.

One of skill in the art will derive insight in producing a eutectic liquid operative in an inventive drug formulation dosage through additional consideration of the following specific systems. Stearic acid and ibuprofen have two eutectic melt temperatures at 62.3° and 63.2° C. at 42 and 65 mole percent ibuprofen, respectively, with these two eutectics bounding an AB₂ complex where A is stearic acid and B ibuprofen corresponding to 59.19 mole percent ibuprofen. Ibuprofen and thymol as detailed above in Stott et al. (J. Controlled Rel. 50:297-308 (1998)) also details a eutectic having a melting point of 32° C. which, while well suited for use in a topical composition, is also appropriate for a soft capsule formulation according to the present invention through encapsulation. Thymol has a melting temperature between 48° and 51° C. By substituting menthol having a melting temperature of 45° C. and an aliphatic ring as opposed to the aromatic ring of thymol, eutectic melting points ranging from 13° to 19° C. based on differences in composition and enantiomeric resolution of the menthol are known. A particularly preferred class of biologically tolerated compounds that form a room or near to temperature liquid eutectic with a pharmaceutically active solid substance is terpenoids. While terpenes such as pinene, limonene and myrcene and including at least site of aliphatic unsaturation are suitable as sigma donors to form a stable complex with an electron donating pharmaceutically active substance, more dramatic complex formation and therefore lower melting temperature eutectics exist for other compounds that occur in volatile oils. Other compounds operative herein for forming a eutectic liquid with a pharmaceutically active substance illustratively include volatile oil alcohols such as benzyl alcohol, borneol, cinnamyl alcohol, citronellol, geraniol, linalool, menthol, phenylethyl alcohol, and terpineol; aldehydes such as anisaldehyde, cinnamaldehyde, benzaldehyde, citral, piperonal or heliotropin, salicylaldehyde, and vanillin; ketones such as carvone, camphor, thujone, and pulegone; esters such as bornyl acetate, methyl salicylate, benzyl benzoate, geranyl acetate, and linalyl acetate; phenols such as thymol, carvacrol, and chavicol; and phenol ethers such as anethol, eugenol, and safrol.

It is appreciated that while a binary eutectic is the simplest to form, ternary, quaternary or even higher order eutectic mixtures are optionally formed. A ternary or higher phase eutectic includes at least one additional pharmaceutically active substance or biologically tolerated compound. Representative examples of additional pharmaceutically active substances and biologically tolerated compounds are those detailed above. A ternary complex formed is typically AₙBₘCₒ where A is a biologically tolerated compound; B is a biologically tolerated compound; C is an additional pharmaceutically active substance or biologically tolerated compound; and n, m and o are each independently integers of one or greater. Preferably n+m+o is between 3 and 5 and more preferably is 3.

While volatile oil components are often susceptible to oxidation in air, as is commonly noted through odor modification upon exposure to the air, this air sensitivity is largely overcome in the present invention through the use of an encapsulating capsule. Thus, volatile oil eutectics that have met with limited acceptance as topical therapeutics are suitable for oral delivery with the proviso that the composition be protected from oxidation within a capsule. Furthermore such volatile eutectics may be included in the overall list of acceptable eutectics for topical administration by virtue of being contained in soft gels in the form of unit dose tubes containing a spout for snip off or twist off to release the contents onto the skin.

In contrast to topical eutectic formulations containing a pharmaceutically active substance in which a low concentration of active substance is of little concern while air oxidation and shelf life stability are problems, according to the present invention the situation is reversed. Preferably, according to the present invention a eutectic such as 30:70 (weight percent/weight percent) ibuprofen:L-menthol having a eutectic melting point at 19° is suitable for encapsulation within a soft capsule, a stable complex corresponds to AB₂ complex where A is ibuprofen with the net result being only a minority of a soft capsule volume being filled with ibuprofen. It is appreciated that a biologically tolerated compound B mixed with the pharmaceutically active substance A to form a room temperature liquid eutectic yielding a stable AB or A₂B complex has a higher percentage of A than AB₂ complexes and therefore is a more compact ingestible form. More preferably, the biologically tolerated compound is itself a liquid at room temperature or has a melting temperature below 46° C.

Capsules operative herein to enclose an inventive eutectic include soft and hard capsules, and variant thereof such as microcapsules. Capsule shells typically include a gelling agent such as gelatin, polyacrylic acid, polyglutamic acid, or any other mammalian, non-mammalian naturally derived hydrocolloid or synthetic gelling agent. Gelling agents used for capsule manufacture are available from established commercial suppliers of pharmaceutical grade gelatins. Gelatin-based capsule manufacture is known to the art as exemplified U.S. Pat. No. 4,935,243.

One or more plasticizers may be incorporated into the capsule shell. Useful plasticizers of the present invention include glycerin, sorbitan, sorbitol, or similar low molecular weight polyols, such as polyethylene glycols, and mixtures thereof.

Capsule shells are prepared by combining appropriate amounts of gelling agent, water, plasticizer, and any optional components in a suitable vessel and agitating and/or stirring while heating until a uniform solution is obtained. Hard gel capsules can then be used for encapsulating the desired quantity of fill material employing methods known to the skilled artisan. Soft shell compositions containing the desired quantity of the fill composition are made by employing standard encapsulation methodology to produce one-piece, hermetically sealed, soft capsules.

The capsules are formed into the desired shape and size for administration, which may be oral, rectal, vaginal or topical. Oral capsules are of a suitable size for easy swallowing and typically contain from about 100 mg to about 2000 mg of the pharmaceutical active composition. Capsules and encapsulation methods are described in P. K. Wilkinson et al., “Softgels: Manufacturing Considerations”, Drugs and the Pharmaceutical Sciences, 41 (Specialized Drug Delivery Systems), P. Tyle, Ed. (Marcel Dekker, Inc., New York, 1990) pp. 409-449.

Other optional ingredients well included in amounts generally known for these ingredients illustratively include: natural or artificial sweeteners; flavoring agents; colorants; antioxidants such as butylated hydroxy anisole or butylated hydroxy toluene; and preservatives such as methyl or propyl paraben, potassium sorbate, or sodium benzoate.

A capsule encapsulated eutectic drug formulation liquid at room temperature is ideally suited for oral delivery. However, it is appreciated that a suitably proportioned capsule dosage is also operative in delivery via routes of topical, rectal, and vaginal. By way of an example, an inventive dosage is readily packaged in a plurality of small capsules held in position against subject skin. The capsules melt at a temperature less than or equal to skin temperature, dissolve in perspiration, or alternatively are concussion rupturable to release a body surface temperature liquid eutectic mixture containing one or more pharmaceutically active substances to contact with the skin. Optionally, an inert backing retains the skin compatible adhesive matrix in contact with the skin while preventing seepage of the pharmaceutically active substance away from the skin surface. Alternatively, it is appreciated that such a plurality of drug formulation dosage capsules are dissolvable in moisture such as that associated with perspiration.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A drug formulation dosage comprising:
an oral medication capsule;
a eutectic liquid at room temperature contained within said capsule comprising: a pharmaceutically active substance solid in pure form at room temperature; and
a biologically tolerated compound forming the eutectic liquid with said active substance.

2. The dosage of claim 1 wherein said pharmaceutically active substance is present at greater than 20 total weight percent of said liquid.

3. The dosage of claim 1 wherein said capsule has a shell comprising gelatin.

4. The dosage of claim 1 wherein said pharmaceutically active substance is ibuprofen.

5. The dosage of claim 1 wherein said pharmaceutically active substance has a melting point in pure form of greater than 40° C.

6. The dosage of claim 1 wherein said pharmaceutically active substance and said biologically tolerated compound form a stable hydrogen bonded complex of the form AB or A₂B or A₃B where A is said pharmaceutically active substance and B is said biologically tolerated compound.

7. The dosage of claim 1 wherein said biologically tolerated compound is a terpene.

8. The dosage of claim 5 wherein said terpene has a moiety selected from the group consisting of: alcohol, aldehyde and ketone.

9. The dosage of claim 1 wherein said biologically tolerated compound is derived from a volatile oil.

10. The dosage of claim 9 wherein said volatile oil is selected from the group consisting of: benzyl alcohol, borneol, cinnamyl alcohol, citronellol, geraniol, linalool, menthol, phenylethyl alcohol, and terpineol; aldehydes such as anisaldehyde, cinnamaldehyde, benzaldehyde, citral, piperonol or heliotropin, salicylaldehyde, vanillan, carvone, camphor, thujone, pulegone, bornyl acetate, methyl salicylate, benzyl benzoate, geranyl acetate, linalyl acetate, thymol, carvacrol, chavicol, anethol, eugenol, safrol, and combinations thereof.

11. The dosage of claim 1 further comprising an excipient selected from the group consisting of: a diluent, a binder, a lubricant and a disintegrant.

12. The dosage of claim 1 wherein said eutectic liquid further comprises at least a second biologically tolerated compound forming at least a three component eutectic with said pharmaceutically active substance and said biologically tolerated compound.

13. The dosage of claim 12 wherein said at least second biologically tolerated compound is a second pharmaceutically active substance.

14. A process of delivering a pharmaceutically active substance that exists as a solid in pure form at room temperature as an oral dosage comprising: administering to a subject orally a pharmaceutically effective amount of said substance as a eutectic liquid formed with a biologically tolerated volatile oil and contained within a capsule disintegrated subsequent to administration to release said substance.

15. The process of claim 14 wherein said substance is present at greater than 20 total weight percent of said liquid.

16. The process of claim 14 wherein said capsule comprises gelatin.

17. The process of claim 15 wherein said substance is ibuprofen.

18. A transdermal medicament delivery system comprising:
a plurality of said drug formulation dosages of claim 1 wherein integrity of said capsule is compromised after exposure to subject skin; and
a skin compatible adhesive matrix in simultaneous contact with said plurality of drug formulation dosages.

19. The system of claim 18 wherein said capsule is a microcapsule.

20. The system of claim 18 wherein said capsule melts at a temperature between 30° and 37° C.

21. The system of claim 18 wherein said capsule dissolves in perspiration.

22. The system of claim 18 wherein said capsule is formed of a material selected from the group consisting of: polyacrylic acid, sugar, gelatin and polyglutamic acid.
